(19) United States
(12) Patent Application Publication
(10) Pub. No.: US 2003/0206936 A1
(43) Pub. Date: Nov. 6, 2003

(54) Cosmetic composition and method of treating skin and hair

(75) Inventors: Scott Sinclair Barclay, Broadstairs (GB); John Anthony Bosley, Kettering (GB); Robert Mark Donovan, Montmorency (FR); Alexander Gordon James, Sharnbrook (GB); Andrew Easson Mayes, Sharnbrook (GB); Julia Sarah Rogers, Sharnbrook (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/420,629

(22) Filed: Apr. 22, 2003

(30) Foreign Application Priority Data
Apr. 22, 2002 (EP) 02252812.9

(51) Int. Cl. A61K 7/135; A61K 7/06; A61K 31/192

(52) U.S. Cl. 424/401; 424/74; 424/62; 514/570

(57) ABSTRACT

A cosmetic composition for topical application to human skin comprising an effective amount of a hydroxy phenyl alkyl carboxylic acid of general formula (I):

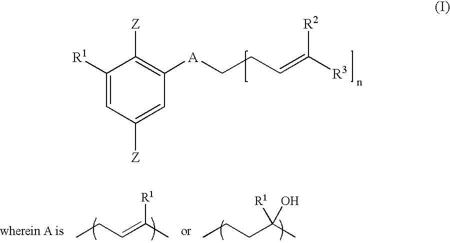

wherein n is an integer between 1 and 5;

wherein z is OH or O or OR¹;

wherein each R¹ is independently C₁₋₄ straight or branched chain alkyl, CH₂OH or COOH;

wherein each R² is independently R¹, CH₂COOH or COOH; and

wherein at least are R² is COOH; and

wherein each R³ is independently —CH₂—, CH₃ or COOH;

and salt and ester derivatives thereof.

This invention relates to a cosmetic composition and method of improving the condition and appearance of skin or hair. In particular, it relates to cosmetic compositions and methods involving certain hydroxy phenyl alkyl derivatives of long chain (e.g. C₁₀-C₁₆) unsaturated carboxylic acids.

Skin in particular is subject to deterioration through dermatological disorders, environmental abuse (wind, air conditioning, central heating) or through the normal ageing process (chronoageing) which may be accelerated by exposure of skin to sun (photoageing). In recent years the demand for cosmetic compositions and cosmetic methods for improving the appearance and condition of skin has grown enormously.

Consumers are increasingly seeking “anti-ageing” cosmetic products which treat or delay the visible signs of chronoageing and photoageing skin such as wrinkles, lines, sagging, hyperpigmentation and age spots.

Consumers also frequently seek other benefits from cosmetic products in addition to anti-ageing. The concept of “sensitive skin” has also raised the consumer demand for cosmetic products which improve the appearance and condition of sensitive, dry and/or flaky skin and to soothe red, and/or irritated skin. Consumers also desire cosmetic products which treat spots, pimples, blemishes etc.

We have now found that effective treatment and prevention of common skin conditions due to chronoageing or photoageing, such as wrinkles, lines, sagging, hyperpigmentation and age spots, may be obtained through the application of cosmetic compositions to the skin which comprise long chain carboxylic acids as described in Claim 1, or derivatives thereof. We have also found that the use of these long chain acids in cosmetic compositions advantageously provides further skin benefits in addition to anti-ageing, such as for soothing sensitive and/or irritated skin, as well as relief for conditions such as dandruff, acne and body odour.

Thus, according to a first aspect of the invention, there is provided a cosmetic composition for topical application to human skin comprising an effective amount of a long chain

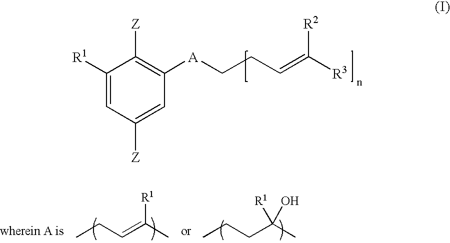

carboxylic acid of structure (I):

wherein n is an integer between 1 and 5;

wherein z is OH or O or OR¹;

wherein each R¹ is independently C₁₋₄ straight or branched chain alkyl, CH₂OH or COOH;

wherein each R² is independently R¹, CH₂ COOH or COOH,

and wherein at least one R² is COOH,

and wherein each R³ is independently —CH₂—, CH₃ or COOH,

and salt and ester derivatives thereof.

Preferably, z is OH. Preferably, R¹ is CH₃. Preferably, n is 3. Preferably, R³ is —CH₂—, although R³ being COOH may be preferred on the terminal repeating unit.

In a further preferred aspect, at least one of the double bonds in the repeating unit of structure (I) has the E configuration; preferably at least two of the double bonds in the repeating unit of structure (I) are E configuration; even more preferably at least three of the double bonds in the repeating unit of structure (I) are E configuration. In a particularly preferred embodiment, all of the double bonds in the repeating unit of structure (I) are E configuration.

Suitable derivatives of the long chain carboxylic acid according to structure (I) are esters (e.g. C₁-C₃₄ esters) and salts thereof especially alkali metal and alkali earth metal salts.

According to a further aspect of the present invention, there is a method of providing at least one cosmetic skin care benefit selected from: treating/preventing wrinkling, sagging, aged and/or photodamaged skin; boosting collagen production in skin, boosting decorin production in skin; soothing irritated, red and/or sensitive skin; improving skin or scalp texture, smoothness or firmness; reducing body odour; reducing or preventing dandruff; and reducing or preventing spots or pimples; the method comprising applying to the skin a topical composition comprising an effective amount of a hydroxy phenyl alkyl carboxylic acid or derivatives thereof according to structure (I).

The present invention also encompasses the use of hydroxy phenyl alkyl carboxylic acid compounds of structure (I) and/or derivatives thereof in a topical composition for providing at least one skin care benefit selected from treating/preventing wrinkling, sagging, aged and/or photodamaged skin; boosting collagen deposition in skin, boosting decorin production in skin; soothing irritated, red and/or sensitive skin; improving skin texture, smoothness or firmness; reducing body odour; reducing or preventing dandruff; and reducing or preventing spots and pimples.

The inventive methods and use of the long chain carboxylic acids of structure (I) may thus provide amongst other benefits anti-ageing benefits, which may result in the promotion of smooth and supple skin with improved elasticity and a reduced or delayed appearance of wrinkles and aged skin, or with improved skin colour. A general improvement in the appearance, texture and condition, in particular with respect to the radiance, clarity, and general youthful appearance of skin may also be achieved.

The inventive methods and uses are also beneficial for soothing and calming sensitive and/or irritated skin. Thus the inventive methods may advantageously provide a wide range of skin care benefits.

These benefits may include improved hydration, texture/tone, smoothness, silkiness, firmness, strength/resilience and radiance.

The term “treating” as used herein includes within its scope reducing, delaying and/or preventing the above mentioned skin conditions such as wrinkled, aged, photodamaged, and/or irritated skin and generally enhancing the quality of skin and improving its appearance and texture by preventing or reducing wrinkling and increasing flexibility, firmness, smoothness, suppleness and elasticity of the skin. The cosmetic methods and the uses of the hydroxy phenyl alkyl acid of structure (I) and/or derivatives according to the invention may be useful for treating skin which is already in a wrinkled, aged, photo-damaged and irritated condition or for treating youthful skin to prevent or reduce those aforementioned deteriorative changes due to the normal ageing/photo-ageing process.

A number of specific long chain carboxylic acids according to structure (I) are particularly suitable. A particularly preferred fatty acid is sargahydroquinoic acid, which as structure (II)

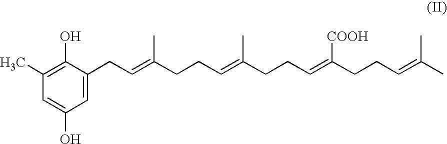

This material has the CA Index name, 12-(2,5-dihydroxy-3-methyl phenyl)-6,10- dimethyl-2- (4-methyl-3-pentyl) -2,6,10 Dodecatrienoic acid.

It has been reported in an extract of brown algae (Sargassum sargarianum var. yezoense)., (Chem. Lett. 1987 (7) 136-6). The acid can be found at levels of 0.6% of an ether extract, together with its oxidation product (two ketone groups on the aromatic ring), which is 86% of the extract.

We have found that this material also exists in extracts of Pycnanthus anolensis nuts, also known as Kombo nuts/seeds (the terms Kombo nut and Kombo seed being synonymous herein), in particular making up approximately 20% of the oil, and being a major constituent of the non-glyceride portion of the oil.

As such, a further preferred aspect of the invention is a cosmetic composition for topical application to human skin comprising an effective amount of kombo nut oil, or its partial glycerides or free fatty acids, which may be derived from partial or total hydrolysis of the oil.

A further suitable long chain carboxylic acid which falls within structure (I) is structure (IV);

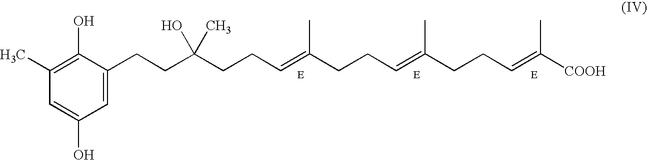

This material is known as chrysochlamic acid. (CA Index name, 16- (2,5-dihydroxy-3-methylphenyl)-14-hydroxy-2,6,10,14-tetramethyl-2,6,10-Hexadecatrienoic acid.

This material may be found in the bark of Chrysochlamys ulei,which may be found in Peru, and its extracts. Extracts from other Chrysochlamys species and other parts of the plant (e.g. leaf, stem, seed or nut) may also be used in the preparation of chrysochlamic acid containing extracts.

In a further preferred aspect of the invention, there is provided a cosmetic composition for topical application to the human skin comprising an effective amount of a Chrysochlamys extract. The extract can be prepared using aqueous, polar or non-polar solvents; particularly preferred are extracts of Chrysochlamys ulei bark.

Compounds according to structure (I) have been found to have a number of important utilities in the composition of the invention; in particular they have been found to be particularly useful in the in vitro assays which have been found to be indicative of cosmetic benefits.

Thus according to a further aspect of the invention, there is provided the use of a compound according to structure (I), or a derivative thereof as a ligand for peroxisome proliferator activated receptor subtype α or γ.

According to another aspect, there is provided the use of a compound according to structure (I) or a derivative thereof as an anti-inflammatory agent.

According to yet another aspect, there is provided the use of a compound according to structure (I) or a derivative thereof to increase the levels of dermal proteins (such as e.g. Decorin) in skin, enhance skin differentiation, or to have an anti-ageing or skin conditioning effect.

According to yet another aspect, there is provided the use of a compound according to structure (I) or a derivative thereof to reduce body (e.g. foot or underarm) odour, or to have an antimicrobial effect.

In the context of reduction of body odour, the concentration of the active compound in the topical composition is sufficient to inhibit catabolism by corynebacteria A, or to have an antimicrobial effect (bacteriostatic or bacteriocidal) upon these bacteria, upon normal use of the composition.

According to yet another aspect, there is provided the use of a compound according to structure (I) or a derivative thereof in a topical composition to reduce sebum production, to prevent or treat acne, or to reduce or prevent pimples and spots.

According to yet another aspect, there is provided the use of a compound according to structure (I) or a derivative thereof as a skin lightening agent, or to prevent skin darkening.

The active according to structure (I) to be employed in accordance with the present invention is present in the topical composition in an effective amount. Normally the total amount of the active is present in an amount between 0.000001% and 20% by weight of the composition. More preferably the amount is from 0.0001% to 5% and most preferably from 0.001% to 1% in order to maximize benefits at minimum cost. In the context of use to prevent malodour, concentrations in the range of 0.001% to 10%, more preferably 0.01% to 5%, more preferably 0.2% to 2% by weight of the topical composition may be preferred.

“Active” in this sense will normally relate to the isolated compounds according to structure (I), though in the context of the use or addition of either kombo nut/seed oil or Chrysochlamys extracts, it may refer to the oil or extract themselves respectively, which are thought to contain relatively high proportions of the compounds according to structure (I).

The composition used according to the invention may also comprise a dermatologically/cosmetically acceptable vehicle to act as a dilutant, dispersant or carrier for the active.

The vehicle may comprise materials commonly employed in skin care products such as water, liquid or solid emollients, silicone oils, emulsifiers, solvents, humectants, thickeners, powders, antioxidants, propellants and the like.

The vehicle will usually form from 5% to 99.99%, preferably from 25% to 80% by weight of the composition, and can, in the absence of other cosmetic adjuncts, form the balance of the composition.

Topical compositions may include vehicles which are liquid at ambient temperature and atmospheric pressure. Hydrophobic liquids may be preferred vehicles, and may include liquid silicones such as liquid polyargonosiloxones (e.g. Dow Canning fluids 344, 345, 244, 245, 246, 556 and the 200 series). Suitable non-silicone hydrophobic liquids include mineral oils, hydrogenated polyisobutene, polyolecene, paraffins, isoparaffins containing at least 10 carbon atoms, and aliphatic or aromatic ester oils such a isopropyl palmitate. Hydrophilic liquid carries such as water may also be employed.

In particular for topical deodorant products, preferred liquid carrier materials can comprise organic solvents. Preferred organic solvents have a melting point of less than 10° C., preferably less than 5° C.; this can benefit both low temperature storage stability and ease of manufacture. The most preferred organic solvents are aliphatic alcohols, in particular those having 2 to 3 carbon atoms, especially ethanol and isopropanol. mixtures of carrier materials may also be used. The total amount of carrier material employed is preferably from 1 to 99% more preferably from 10% to 98%, and most preferably from 50% to 97% by weight of the composition, excluding any volatile propellant that might also be present.

A variety of other materials may also be employed in the compositions of the invention. In certain aspects of the invention, an additional deodorant active may be desirable. This might be a perfume, an antiperspirant active, or an antimicrobial active. Levels of perfume incorporation are preferably up to 4% by weight, particularly form 0.1% to 2% by weight, and especially from 0.7% to 1.7% by weight of the composition.

Typical antiperspirant actives include astringent active salts, in particular, aluminium, zirconium and mixed aluminium/zirconium salts, including both inorganic salts, salts with organic anions and complexes. Preferred astringent salts include aluminium, zirconium and aluminium/zirconium halides and halohydrate salts, such as chlorohydrates. Preferred levels of incorporation are from 0.5% to 60%, particularly from 5% to 30% or 40% and especially from 5% or 10% to 30% or 35% by weight of the composition. Typical antimicrobial actives include 2′,4,4′-trichloro, 2-hdyroxy-diphenyl ether (triclosan), and 3,7,11-trimethyldodeca-2,6,10-trienol (farnesol). Typical levels of incorporation are from 0.01% to 1%, in particular from 0.03% to 0.5%, or especially from 0.05% to 0.3% by weight of the composition.

Structurants and emulsifiers are further additional components of the compositions of the invention that are highly desirable in certain product forms. Structurants, when employed, are preferably present at from 1% to 30% by weight of the composition, whilst emulsifiers are preferably present at from 0.1% to 10% by weight of the composition. Suitable structurants include cellulosic thickeners such as hydroxypropylcellulose and hydroxyethylcellulose. Emulsion pump sprays, roll-ons, creams, and gel compositions according to the invention can be formed using a range of oils, waxes and emulsifiers. Suitable emulsifiers include steareth-20, ceteareth-20, glyceryl stearate, cetyl alcohol, PEG-20 stearate, and dimethicone copolyol. Suspension aerosols, roll-ons, sticks, and creams require structurants to slow sedimentation (in fluid compositions) and to give the desired product consistency to non-fluid compositions.

Suitable structurants include sodium stearate, stearyl alcohol, hydrogenated castor oil, synthetic waxes, paraffin waxes, hydroxystearic acid, quaternium-18 bentonite, quaternium-18 hectorite, and silica. Some of the above materials also function as suspending agents in certain compositions.

Further emulsifiers desirable in certain compositions of the invention are perfume solubilisers and wash-off agents. Examples of the former include PEG-hydrogenated castor oil, preferably present at up to 1.5% by weight, more preferably 0.3 to 0.7% by weight. Examples of the latter include poly(oxyethylene) ethers. Sensory modifiers are further desirable components in certain compositions of the invention. Such materials are preferably used at a level of up to 20% by weight of the composition. Other additional components that may also be included are colourants and preservatives, for example C₁-C₃ alkyl parabens.

The vehicle will usually form from 5% to 99.99% or more, preferably from 25% to 80% by weight of the composition, and can, in the absence of other cosmetic adjuncts, form the balance of the composition.

Besides the active other specific skin-benefit actives such as sunscreens, skin lightening agents and skin tanning agents may also be included. The vehicle may also further include adjuncts such as perfumes, opacifiers, preservatives, colourants and buffers.

To prepare the topical composition used in the method of the present invention, the usual manner for preparing skin care products may be employed. The active components are generally incorporated in a dermatologically acceptable carrier in a conventional manner. The active components can suitably first be dissolved or dispersed in a portion of the water or another solvent or liquid to be incorporated in the composition, before the final topical composition is prepared. The preferred compositions are oil-in-water or water-in-oil emulsions.

skin-care product such as a cream, gel, soft solid or lotion or the like. The composition can also be in the form of a so-called “wash-off” product e.g. a bath or shower gel, or a shampoo possibly containing a delivery system for the actives to promote adherence to the skin during rinsing. Most preferably the product is a “leave-on” product; that is, a product to be applied to the skin without a deliberate rinsing step soon after its application to the skin.

The composition may be packaged in any suitable manner such as in a jar, a bottle, tube, roll-ball, pump spray, squeeze spray, cosmetic or deodorant stick, or aerosol, wipes the like, in the conventional manner. The composition may also be encapsulated in a “unit dose” form such as a capsule, or micro-encapsulated.

The method of the present invention may be carried out one or more times daily to the skin or hair which requires treatment. Improvements in skin appearance will usually become visible after 1 to 3 months, depending on skin condition, the concentration of the active components used in the inventive method, the amount of composition used and the frequency with which it is applied. In general, a small quantity of the composition, for example from 0.1 to 5 ml is applied to the skin or hair from a suitable container or applicator and spread over and/or rubbed into the skin or hair using the hands or fingers, or a suitable device. A rinsing step may optionally follow depending on whether the composition is formulated as a “leave-on” or a “rinse-off” product.

In order that the present invention may be more readily understood, the following examples are given, by way of illustration only, with reference to the accompanying drawings, in which:

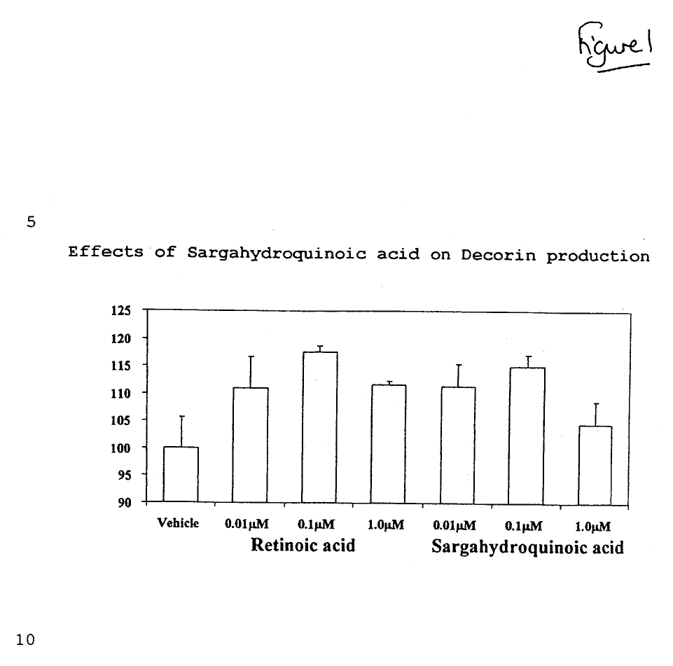

FIG. 1 shows the effect of sargahydroquinoic acid on decorin synthesis;

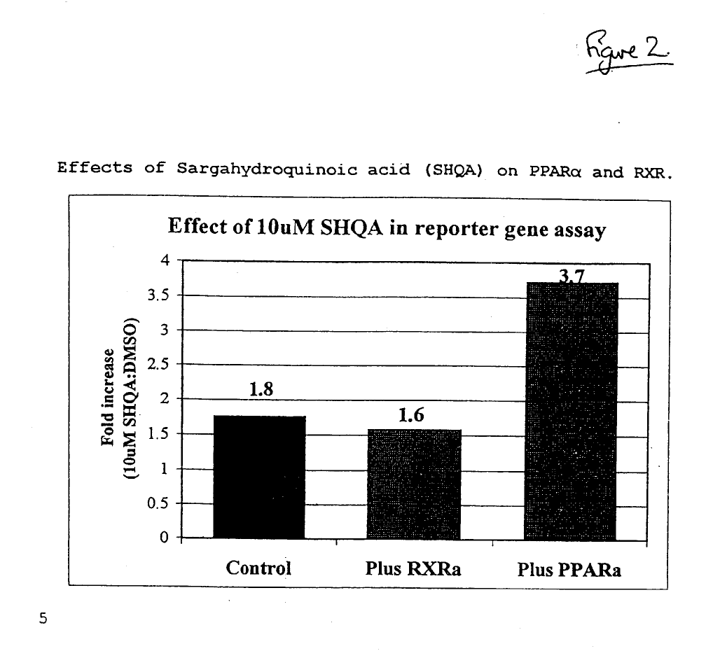

FIG. 2 shows the effect of sargahydroquinoic acid in a reporter gene assay;

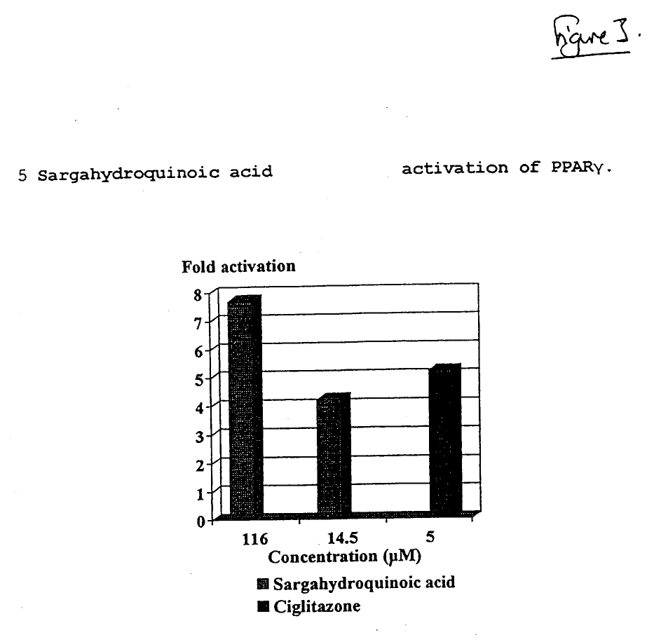

FIG. 3 shows the effect of sargahydroquinoic acid on PPAR γ activation; and

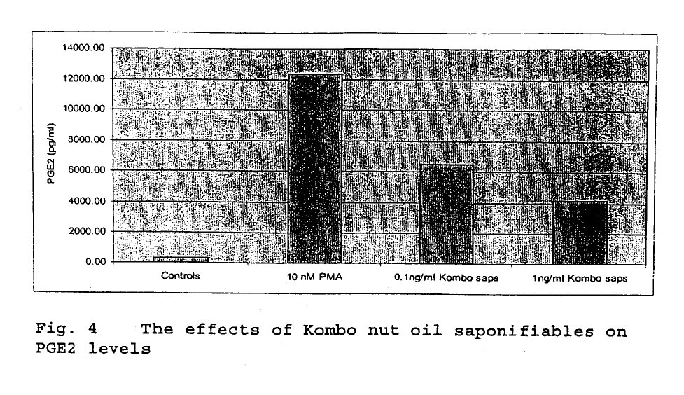

FIG. 4 shows the effect of kombo nut oil saponifiables on PGE2 levels

### EXAMPLES

It is known from our co-pending European application no. 99908956.8 that topical retinoic acid treatments can be used to cause upregulation of procollagen I and decorin in vivo. To this end, the passages under the heading “Identification of procollagen I and decorin upregulation in skin in vivo following topical retinoic acid treatment for comparative purposes” in that application are incorporated herein in their entirety.

### Example 1

The first example demonstrates the anti-ageing benefits of sargahydroquinoic acid.

Procedure For Measuring Procollagen-I and Decorin Synthesis in Human Dermal Fibroblasts

Preparation of Dermal Fibroblast Conditioned Medium

Primary human foreskin fibroblasts at passage 2 (P2) were seeded into 12-well plates at 10000 cells/cm² and maintained for 24 hours in an atmosphere of 5% carbon dioxide and 4% oxygen in Dulbeccos Modified Eagles Medium (DMEM) supplemented with 10% foetal calf serum. After this time the cells were washed with serum free DMEM and then incubated in fresh serum free DMEM for a further 60 hours. The fibroblast monolayers were then washed again with serum free DMEM. Test reagents (retinoic acid and Sargahydroquinoic acid, isolated from Kombo nut oil at >90% purity) and vehicle controls were added to the cells in triplicate in a final volume of 0.4ml/well fresh serum free DMEM and incubated for a further 24 hours. This fibroblast conditioned medium was either analyzed immediately or snap frozen in liquid nitrogen and stored at −70° C. for future analysis. The cells were then counted and data from the dot-blot analysis subsequently standardized to cell number.

Dot Blot Assay for Procollagen-I and Decorin Protein in Dermal Fibroblast Conditioned Medium

Samples of conditioned medium from dermal fibroblasts treated with vehicle (as a control) or test reagents were supplemented with 20 mM dithiothreitol (1:10 dilution of 200 mM stock solution) and 0.1% sodium dodecylsulphate (1:100 dilution of 10% stock solution), mixed well and then incubated at 75° C. for 2 minutes. A standard for the assay was generated by serial dilution of neat fibroblast conditioned medium from fibroblasts seeded at 10000 cells/cm² in a 175cm² flask and maintained in serum free DMEM as described above.

Assay samples were subsequently applied in triplicate to a pre-wetted sheet of Immobilon-P transfer membrane using the 96-well Bio-Dot Apparatus from Bio-Rad as described in the manufacturer's guidelines. Approximately 200 μl of medium was applied per well. The medium was allowed to filter through the membrane under gravity (30 minutes) after which the membrane was washed twice with PBS (200 μl ). These PBS washes were allowed to filter through the membrane under gravity (2×15 minutes). The Bio-Dot apparatus was then attached to a vacuum manifold and a third and final PBS wash carried out under suction. The apparatus was disassembled, the membrane removed and quickly cut as required before being placed in blocking buffer overnight at 4° C.

Membranes prepared for decorin analysis were blocked with 3% (w/v) BSA/0.1% (v/v) Tween 20 in PBS, whilst those for procollagen-I analysis were blocked with 5% (w/v) non fat dried milk powder/ 0.05% Tween 20 in PBS. The following day, the membranes were probed with 1:10000 dilution of primary antibodies to either human procollagen-I (MAB1912; rat monoclonal; Chemicon Int. Inc., Temecula, Calif.) or human decorin (rabbit polyclonal; Biogenesis) for 2 hours at room temperature. The membranes were subsequently washed with TBS/ 0.05% Tween 20 (3×5 minutes) and then incubated with 1:1000 dilution of ¹²⁵I-conjugated anti-rat or anti-rabbit F(ab′)2 fragments (Amersham) as required for 1 hour at room temperature.

Following this the Immobilon strips were again washed with TBS/Tween 20 (3×5 minutes) before being allowed to dry in air at room temperature. The dried membranes were wrapped in cellophane and exposed to a Molecular Dynamics storage phosphor screen for 16-18 hours. At the end of this time the exposed screen was scanned by a phosphorimager (Molecular Dynamics Phosphorimager SF) using ImageQuant™ software. Dot intensity was assessed by computer-assisted image TM analysis using the quantification tools in ImageQuant™, standardized to cell number and the effects of various test reagents on decorin and procollagen-I synthesis were determined relative to a vehicle treated control value of 100 arbitrary units.

The results of the assays performed with sargahydroauinoic acid and retinoic acid are shown graphically in FIG. 1. The level of decorin in skin is associated with improved condition and appearance of skin. Increasing the level of decorin in skin is important for controlled and correct deposition of collagen in skin which is associated with many skin benefits such as wrinkle effacement and dermal repair of photodamaged skin. Sargahydroquinoic acid enhances decorin production, and this effect is similar to that observed for retinoic acid.

### Example 2

### Reporter Gene Assay (PPARα)

PPARα ligands are known to provide a number of cosmetic benefits when applied to skin as topical compositions. These include not only boosting skin condition and having anti-ageing effects, but they also have a role in hair follicle cycling and growth, homeostasis of proliferation and differentiation in the epidermis and the skin's immune system. PPARα ligands can also boost skin differentiation, and decrease the time for barrier repair, as well as boost barrier lipid levels and lamellar granule synthesis.

The following assay protocol is responsive to ligands of PPARα or retinoid X receptor. The data indicates that sargahydroquinoic acid is a ligand for PPARα.

Reporter Gene Assay Protocol

Cos-7 cells (ECACC No. 87021302) were routinely grown in DMEM with 10% FCS (foetal calf serum) at 37° C., 5% CO₂ to 80% confluency. Transient transfections were performed as described by the manufacturers (GibcoBRL). Briefly, cells were plated out in 24 well plates at 50,000 cells per well and incubated overnight in DMEM with 10% FCS at 37° C., 5% CO₂,. Cells were then transfected using the LipofectAMINE reagent. For each well, 0.5 μg of DNA mix (for “control” cells pPPRE₃TK-luc 0.40 μg; pRL-TK 0.04 μg; pcDNA3.1(−) 0.03 μg; pRSV 0.03 μg; for “plus RXRα” cells pPPRE₃TK-luc 0.40 μg; pRL-TK 0.04 μg; pcDNA3.1(−) 0.03 μg; pRSV/RXRα 0.03 μg; for “plus PPARα” cells pPPRE₃TK-luc0.40 μg; pRL-TK 0.04 μg pcDNA3.1(−)/PPARα 0.03 μg pRSV 0.03 μg) in 25 μl of DMEM was incubated with 1 μl LipofectAMINE, also in 25 μl of DMEM for 45 minutes.

The mixture was then made up to 250 μl per well and added to the cells, which had been washed with 1 ml of DMEM. Cells were then incubated for 5 hours at 37° C., 5% CO₂, and 250 μl DMEM with 20% SBCS (charcoal stripped bovine calf serum (Sigma)) added. Cells were allowed to recover for 18 hours at 37° C., 5% CO₂ before being treated. The transfection mix was removed from the cells and replaced with treatment mix (DMSO or 10 μM Sargahydroquinoic acid, isolated from Kombo nut oil at >90% purity) and incubated for 24 hours at 37° C., 5% CO₂. Sargahydroquinoic acid was made up as 10 mM stock in DMSO and diluted 1000-fold into DMEM containing 10% SBCS (500 μl per well) immediately before being added to the cells.

Each treatment was performed in triplicate. Cells were then washed with 1 ml of PBS (without calcium or magnesium) and lysed with 100 μl per well of 1× Passive Lysis Buffer (as supplied with Promega Dual Luciferase assay kit). Lysis was allowed to continue for 15 minutes and then the lysate was assayed for Firefly and Renilla luciferase activity using the Promega Dual Luciferase assay kit. For the assay 20 μl of lysate was taken and assayed as described in the kit instructions using a MLX microtiter plate luminometer (Dynex).

The results are shown graphically in FIG. 2.

### Example 3

Reporter Gene Assay (PPARγ)

Peroxisome proliferator activated receptor gamma (PPARγ) is a nuclear hormone receptor which forms part of the PPAR group of transcription factors. These are ligand activated transcription factors which bind DNA in a heterodimeric complex with a second nuclear hormone receptor retinoid X receptor. PPARγ is known to be expressed in human keratinocytes and the epidermis and PPARγ ligands are known to inhibit the proliferation of normal and psoriatic human keratinocytes in culture. This will give benefits in terms of reducing/preventing hyperproliferative dry skin conditions such as psoriasis, and should enhance the condition of skin.

Reporter Gene Assay Protocol

This assay is based on that described by Kliewer et al (Nature 358 771-774 1992). Cos-7 cells (ECACC No. 87021302) were seeded in 24-well plates at a density of 0.325×10⁵ cells/sell. Cells were grown overnight at 37° C./5% CO₂ in DMEM containing 10% FCS, 2 mM L-glutamine, 100 iu/ml penicillin and 100 μg/ml streptomycin. Cells were washed with transfection media (DMEM containing 2 mM L-glutamine) then transiently transfected with 4 plasmids: a PPAR-responsive firefly luciferase reporter gene (pPPRE3TK-luc); mammalian expression plasmids (pcDNA3/hPPARγl and pRSV/hRXRα) containing human PPARγl and RXRα cDNAs respectively and a control plasmid (PRLTK, Promega) which constitutatively expresses the renilla luciferase gene. Transfection was performed using Lipofectamine (Gibco Brl) as directed by the manufacturers.

Transfected cells were incubated for 6 h at 37° C./5% CO₂ in DMEM and then for a further 46 hours in the presence or absence of sargahydroquinoic acid (isolated from Kombo nut oil at >90% purity). After 46 hours cell lysates were prepared and the level of firefly and renilla luciferase determined using the Dual luciferase assay system (Promega) and a MLX microtitre plate luminometer (Dynex). The level of firely luciferase (normalised against the renilla luciferase control) provides a measure of reporter gene activity, which in turn reflects the level of PPARγ activation.

The results of this assay are shown in FIG. 3.

The reporter gene assay data also supports skin differentiation, anti-inflammatory and prevention and treatment of acne benefits.

### Example 4

This example demonstrates the effect of sargahydroquinoic acid on microbial number and biotious formations, in particular in the context of axillary (underarm) malodour.

It is known that a fatty acid catabolizing sub-group of the Corynebacherium genes, termed corynebacteria A, contributes strongly to the formation of axillary malodour (see for example WO 00/01353 and WO 00/01359).

An in vitro model system, reproducing fatty acid catabolism by corynebacteria A, consisted of 250 ml baffled shake flasks, to which were added 30 ml semi-synthetic medium (see below) supplemented with fatty acid substrate (2.0 mg/ml pentadecanoic acid) and non-fatty acid substrate (0.5 mg/ml glucose). This system was employed to evaluate sargahydroquinoic acid as a potential deodorant active: to each flask (other than the control) was added and specific dose of sargahydroquinoic acid (isolated from Kombo nut oil at >90% purity), from a 25 g/l stock emulsion in semi-synthetic medium supplemented with 5.0 mg/ml Gum Arabic (formed by ultra-homogenisation at 24000 rpm for 1 min) Flasks were inoculated with fresh bacterial biomass (Corynebacterium A NCIMB 40928), pre-grown for 24 h in TSBT (see below), to give starting optical densities (A_{5 9 0}) of ˜1.0. Following inoculation, flasks were incubated aerobically at 35°0 C., with agitation (130 rpm), and analysed at 24 h. Culture viability was determined by total viable count (TVC) analysis on TSAT plates (see below) following serial dilution in quarter-strength Ringers solution. Fatty acid concentrations were determined by capillary gas chromatography (GC) (see below), while residual glucose levels were measured with blood glucose test strips (BM-Test 1-44; Boehringer Mannheim) used in conjunction with a ReflofluxS glucose meter (Boehringer Mannheim).

A ‘sub-lethal’ effect is defined as significant inhibition of fatty acid catabolism, typically greater than 50% inhibition of pentadecanoic acid utilization, without concomitant reductions in Corynebacterium A viability (≦1 log₁₀ CFU/ml reduction) or glucose utilization (≦10% reduction). Outside one or both of these defined boundaries, the effect may be described as ‘antimicrobial’, where ‘antimicrobial’ includes both bacteriostatic & bactericidal effects.

Composition of Tween-supplemented Tryptone soya broth & agar (TSBT & TSAT) used for growth & maintenance of axillary bacterial (g/l): Tryptone soya broth (30.0); Yeast Extract (Beta Lab) (10.0); Tween 80™ (1.0); ± Agar (20.0).

Composition of semi-synthetic medium used in in vitro system simulating fatty acid catabolism by corynebacteria A (g/l): KH₂PO, (1.6);(NH₄) ₂HPO₄(5.0); Na₂SO₄ (0.38); Yeast Nitrogen Base (Difco)(3.35); Yeast Extract (Beta Lab)(0.5); Tween 80™ (0.2); Triton X-100™ (0.2); MgCl₂.6H₂0(0.5).

Fatty acid levels in the flasks were determined by capillary GC analysis. Initially, 5.0 ml aliquots from each flask were transferred into universal tubes; an internal standard (lauric acid, 1.0 mg/ml final concentration) was added to each tube and the culture medium acidified (pH ˜2) by the addition of hydrochloric acid. Liquid-liquid extraction was then carried out using 2 vol (10 ml) ethyl acetate; organic and aqueous phases were resolved by centrifugation (2000 rpm, 3 min). ˜0.75 ml of each organic (upper) phase was then transferred to a sampling vial prior to analysis on a Perkin Elmer 8000 (Series 2) GC fitted with a 15 m ×0.32 mm (internal diameter) FFAP (nitroterephalic acid modified PEG/siloxane copolymer) fused silica capillary column (film thickness 0.25 mm) (Quadrex).

This column was attached to the split-splitless injector and flame ionisation detector (FID) of the GC; injector and detector temperatures were each 300° C. Carrier gas for the column was helium (6 psi), while hydrogen (17 psi) and air (23 psi) supplied the FID. The temperature programme for fatty acid analysis was 80° C.(2 min); 80-250° C. (20° C./min); 250° C. (5 min). Sample size for injection was 0.5-1.0 μl . Fatty acid levels in the flasks were quantified by comparison of peak areas with known levels of both internal (lauric acid) and externally-run (pentadecanoic acid) standards.

Results

| | | | Pentadecanoic Acid | |
|---|---|---|---|---|
| | | Glucose | | Inhibition of |
| [Sargahydroquinoic | TVC (log₁₀ | Utilisation | Utilisation | Utilisation |
| acid] (g/l) | CFU/ml) | (%) | (%) | (%) |
| 0.0 | 8.40 | 100 | 100 | 0 |
| 0.05 | 8.67 | 100 | 99 | (1) |
| 0.1* | 8.83* | 100* | 58* | 42* |
| 0.25* | 7.93* | >90* | 15* | 85* |
| 0.5* | 7.85* | 100* | 19* | 81* |
| 1.0 | 7.3 | >90 | 5 | 95 |
| 2.5 | <3.0 | 7 | 25 | 75 |

The figures indicated * denote a sub-lethal inhibition of pentadecanoic acid utilization. Other effects are due to antimicrobial (bacteriostatic or bacteriocidal) inhibition or, where in paretheses, are not significant.

### Example 5

The anti-inflammatory effects were determined by in vitro tests wherein the production of Prostaglandin E2 (=PGE2) production by the human skin fibroblasts is measured after being induced by the inflammatory modulus Phorbol myristate acetate (PMA). A reduction in the levels of PGE2 is indicative for the anti-inflammatory effect. Primary human foreskin fibroblasts at passage 2 (P2) were seeded into 96-well plates at 10000 cells/well and maintained for 24 hours in an atmosphere of 5% carbon dioxide in Dulbeccos Modified Eagles Medium (DMEM) supplemented with 10% foetal calf serum. Kombo nut oil saponifiables (containing 20% sargahydroquinoic acid) were added to fresh cell media in ethanol (final concentration 1%) in triplicate and incubated for a further 24 hours. PMA in ethanol/cell media was added to the media and the cells incubated for a futher 24 hours. PMA represents an external stressor which induces oxidative stress and inflammatory responses in cells. The fibroblasts / media were then analysed as described below immediately or snap frozen in liquid nitrogen and stored at −70° C. for future analysis.

Prostaglandin E2 (PGE2) Assay

Volumes of 50 μl culture -medium were taken for PGE2 assay after gently shaking the culture plate. PGE2 levels in the medium were determined with a Biotrak PGE2 immunoassay kit (Amersham, UK). The assay is based on the competition between unlabelled PGE2 in the sample and a fixed quantity of horseradish peroxidase labelled PGE2 for a limited amount of fixed PGE2 specific antibody. Concentrations of unlabelled sample PGE2 are determined according to a standard curve which was obtained at the same time.

The effects of Kombo nut oil saponifiables on PGE2 levels are shown in FIG. 4. The data demonstrates that challenging fibroblast cells with an inflammatory stimulus such as PMA causes an increase in the inflammatory response as measured by PGE2 production. Kombo nut oil saponifiables (saps), which contain sargahydroquinoic acid, even at the levels of 0.ng/ml, dramatically reduces the inflammatory response as measured by PGE2 production. —good anti-inflammatory activity.

### Example 6

The formulation below describes an oil in water emulsion cream suitable for the methods and uses according to the present invention. The percentages indicated are by weight of the composition.

| Ingredient | wt % |
|---|---|
| Mineral Oil | 4 |
| Chrysochlamys bark extract | 1.15 |
| Brij 56* | 4 |
| Alfol 16RD* | 4 |
| Triethanolamine | 0.75 |
| Butane-1,3-diol | 3 |
| Xanthan gum | 0.3 |
| Perfume | Qs |
| Butylated hydroxy toluene | 0.01 |
| Water | To 100 |

*Brij 56 is cetyl alcohol POE (10)

Alfol 16RD is cetyl alcohol

### Example 7

The formulation below describes an emulsion cream according to the present invention.

| FULL CHEMICAL | | |
|---|---|---|
| NAME OR CTFA NAME | TRADE NAME | WT. % |
| Sarghydroquinoic Acid | | 2.0 |
| Disodium EDTA | Sequesterene Na2 | 0.05 |
| Magnesium aluminium | Veegum Ultra | 0.6 |
| silicate | | |
| Methyl paraben | Methyl Paraben | 0.15 |
| Simethicone | DC Antifoam Emulsion | 0.01 |
| Butylene glycol 1,3 | Butylene Glycol 1,3 | 3.0 |
| Hydroxyethylcellulose | Natrosol 250HHR | 0.5 |
| Glycerine, USP | Glycerine USP | 2.0 |
| Xanthan gum | Keltrol 1000 | 0.2 |
| Triethanolamine | Triethanolamine (99%) | 1.2 |
| Stearic acid | Pristerene 4911 | 3.0 |
| Propyl paraben NF | Propylparaben NF | 0.1 |
| Glyceryl hydrostearate | Naturechem GMHS | 1.5 |
| Stearyl alcohol | Lanette 18 DEO | 1.5 |
| Isostearyl palmitate | Protachem ISP | 6.0 |
| C₁₂₋₁₅ alcohols | Hetester FAO | 3.0 |
| octanoate | | |
| Dimethicone | Silicone Fluid 200 | 1.0 |
| | (50 cts) | |
| Cholesterol NF | Cholesterol NF | 0.5 |
| Sorbitan stearate | Sorbitan Stearate | 1.0 |
| Butylated | Embanox BHT | 0.05 |
| hydroxytoluene | | |
| Tocopheryl acetate | Vitamin E Acetate | 0.1 |
| PEG-100 stearate | Myrj 59 | 2.0 |
| Sodium stearyl | Pationic SSL | 0.5 |
| lactylate | | |
| Hydroxycaprylic acid | Hydroxycaprylic Acid | 0.1 |
| Retinyl palmitate | Vitamin A Palmitate | 0.06 |
| Alpha-bisabolol | Alpha-bisabolol | 0.2 |
| Water, DI | | q.s. to 100 |

Both the above topical compositions of example 5 and 6 may provide an effective cosmetic treatment to improve the appearance of wrinkled, aged, photo-damaged, and/or irritated skin, when applied to skin that has deteriorated through the ageing or photoageing or when applied to youthful skin to help prevent or delay such deteriorative changes. The compositions can be processed in a conventional manner.

### Examples 8 to 16

Examples 8 to 16 represent suitable underarm topical compositions according to the invention.

TABLE 1

| | Example | | | | | |
|---|---|---|---|---|---|---|
| | 8 | 9 | 10 | 11 | 12 | 13 |
| CAP 40¹ | 85 | 35 | 85 | 35 | 85 | 35 |
| Ethanol (96%) | 0 | 61.17 | 0 | 62.67 | 0 | 62.67 |
| DC 245² | 6.9 | 0 | 8.4 | 0 | 8.4 | 0 |
| AACH³ | 5 | 0 | 5 | 0 | 5 | 0 |
| Sargahydroquinoic | 0 | 0 | 0.5 | 0.5 | 0 | 0 |
| acid | | | | | | |
| Kombo nut oil | 2.0 | 2.0 | 0 | 0 | 0 | 0 |
| Chrysochlamys ulei | 0 | 0 | 0 | 0 | 0.5 | 0.5 |
| bark extract | | | | | | |
| Bentone 38⁴ | 0.5 | 0 | 0.5 | 0 | 0.5 | 0 |
| Perfume | 0.6 | 1.5 | 0.6 | 1.5 | 0.6 | 1.5 |
| Isopropyl myristate | 0 | 0.33 | 0 | 0.33 | 0 | 0.33 |

1. Mixture of butane, isobutane and propane, ex Calor.

2. Cyclomethicone, ex Dow Corning.

3. Activated aluminium chlorohydrate, grade A296, ex 5 Giulini.

4. Quaternium-18 hectorite, ex Rheox.

TABLE 2

| Example | 14 | 15 | 16 |
|---|---|---|---|
| Ethanol | 59.4 | 0 | 70 |
| Water | 39.6 | 0 | 27.85 |
| Sargahydroquinoic acid | 1.0 | 1.0 | 1.0 |
| Cremaphor RH40¹ | 0 | 0 | 0.5 |
| Klucel M² | 0 | 0 | 0.65 |
| AAZG³ | 0 | 25 | 0 |
| DC 245 | 0 | 50.8 | 0 |
| Stearyl alcohol | 0 | 14 | 0 |
| Superfino talc | 0 | 3.2 | 0 |
| PEG-8 distearate | 0 | 1 | 0 |
| Castor wax MP80 | 0 | 4 | 0 |
| Perfume | 0 | 1 | 0 |

¹PEG-hydrogenated castor oil, ex BASF.
²Hydroxypropylcellulose, ex Aqualon.
³Aluminium zirconium tetrachlorohydrex-glycine, Q5-7167, ex Summit.

All percentages are by weight.

Compositions 8 to 13 are propellant driven aerosol compositions, example 14 is a pump spray.

Composition example 15 is an antiperspirant stick, and example 16 is a roll-on composition.

### Example 17-18

The following is an example of a shampoo composition according to the invention.

| Ingredient | Example 17 |
|---|---|
| Chemical Name | Active ingredient weight % |
| SLES 2EO | 14 |
| Cocoamidopropylbetaine | 2 |
| Guar hydroxypropyltrimonium | 0.1 |
| chloride | |
| Dimethiconol | 1 |
| Crosslinked polyacrylic acid | 0.4 |
| Zinc pyrithione | 0.6 |
| Lactic acid | 0.5 |
| Mica + titanium dioxide | 0.2 |
| Sargahydroquinoic acid | 2.0 |
| Sodium benzoate | 0.5 |
| Water | to 100 |

| Ingredient | Example 18 |
|---|---|
| Chemical name | Active ingredient weight % |
| SLES (2EO) | 16 |
| CAPB | 2 |
| Kombo nut oil or | 10 |
| Sargahydroquinoic acid | |
| Carbopol TM | 0.4 |
| BHT | 0.024 |
| Water | 71.576 |

### Examples 19 and 20

The following is an example of a rinse-off conditioning composition according to the invention.

| Ingredient | Example 19 |
|---|---|
| Chemical Name | weight % |
| Soft water | q.s. to 100 |
| Citric acid (50% aqueous) | 0.185 |
| Stearamidopropyldimethylamine | 0.5 |
| Propylene glycol | 0.5 |
| Dicetyldimonium chloride/PG | 2.1 |
| Zinc pyrithione | 0.4 |
| Lactic acid | 0.1 |
| Sargahydroquinoic acid | 2.0 |
| TAPS | 0.1 |
| Stearyl alcohol and | 1.0 |
| ceteareth-20 blend | |
| Cetyl alcohol | 3.25 |
| Disodium EDTA | 0.1 |
| Methylchloroisothiazoline/ | 0.05 |
| methylisothiazoline | |
| DMDM hydantoin | 0.1 |
| Potassium hydroxide (50% | 0.033 |
| aqueous) | |
| Fragrance | 0.2 |
| Potassium chloride | 0.1 |

| Ingredient | Example 20 |
|---|---|
| Chemical Name | weight % |
| Soft water | q.s. to 100 |
| Citric acid (50% aqueous) | 0.185 |
| Stearamidopropyldimethylamine | 0.5 |
| Propylene glycol | 0.5 |
| Dicetyldimonium chloride/PG | 2.1 |
| Sargahydroquinoic acid | 2.0 |
| Stearyl alcohol and ceteareth-20 blend | 1.0 |
| Cetyl alcohol | 3.25 |
| Disodium EDTA | 0.1 |
| Methylchloroisothiazoline/ | 0.05 |
| methylisothiazoline | |
| DMDM hydantoin | 0.1 |
| Potassium hydroxide (50% aqueous) | 0.033 |
| Fragrance | 0.2 |
| Potassium chloride | 0.1 |

1. A cosmetic composition for topical application to human skin comprising an effective amount of a hydroxy phenyl 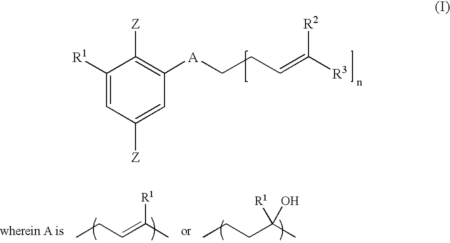
alkyl carboxylic acid of general formula (I):
wherein n is an integer between 1 and 5;
wherein z is OH or O or OR¹;
wherein each R¹ is independently C₁₋₄ straight or branched chain alkyl, CH₂OH or COOH;
wherein each R² is independently R¹, CH₂COOH or COOH, and wherein at least one R² is COOH;
wherein each R³ is independently —CH₂—, CH₃ or COOH; and salt and ester derivatives thereof.

2. A cosmetic composition according to claim 1, wherein R¹ is methyl.

3. A cosmetic composition according to claim 1, wherein Z is OH.

4. A cosmetic composition according claim 1, wherein n is 3.

5. A cosmetic composition according to claim 1, wherein R³ is —CH₂—.

6. A cosmetic composition according to claim 1, wherein at least one of the double bonds in structure (I) has the E configuration.

7. A cosmetic composition according to claim 6 wherein all of the double bonds have the E configuration.

8. A cosmetic composition according to claim 1, wherein the derivative of structure (I) is a C₁₋₃₄ ester or a salt.

9. A cosmetic composition according to claim 1, wherein the hydroxy phenyl alkyl carboxylic acid is sargahydroquinoic acid.

10. A cosmetic composition according to claim 1, wherein the hydroxy phenyl alkyl carboxylic acid is chrysochlamic acid.

11. A cosmetic composition according claim 1, wherein the cosmetic composition contains an extract of kombo nut/seed oil or its partial glycerides or free fatty acids.

12. A cosmetic composition according to claim 1, containing an extract of Chrysochlamys.

13. A cosmetic composition according to claim 1, wherein the composition is a deodorant composition.

14. A method of providing at least one skin care benefit selected from: treating/preventing wrinkling, sagging, aged and/or photodamaged skin; boasting collagen deposition on skin, boosting decorin production in skin; soothing irritated, red and/or sensitive skin; improving skin texture, smoothness and/or firmness; providing anti-inflammatory benefits; enhancing skin differentiation; reducing body odour; reducing sebum production; the prevention or treatment of acne; or as an agent to promote skin lightening, or prevent skin darkening; the method comprising applying to the skin a topical composition comprising an effective amount of a hydroxy phenyl alkyl carboxylic acid according to any of the preceding claims.
